SEQUENCE LISTING (2 sequences)

SEQ ID NO: 1 - protein, 13 aa, Homo Sapiens
CNGRCVSGCAGRC

SEQ ID NO: 2 - protein, 5 aa, Artificial Sequence
  DOMAIN (1)...(5): Synthetic Peptide (Cyclic)
RGDXV

(19) United States
(12) Patent Application Publication
(10) Pub. No.: US 2002/0044936 A1
(43) Pub. Date: Apr. 18, 2002

(54) Method of inhibiting angiogenesis and tumor growth and preventing tumor growth and metastases

(75) Inventors: Barry Coller, New York, NY (US); Robert Jordan, Malvern, PA (US); Judith Varner, Encinitas, CA (US)

Correspondence Address: BAKER & BOTTS, 30 ROCKEFELLER PLAZA, NEW YORK, NY, 10112

(73) Assignee: MOUNT SINAI SCHOOL OF MEDICINE OF NEW YORK UNIVERSITY, CENTOCOR INC.

(21) Appl. No.: 09/855,886

(22) Filed: May 15, 2001

Related U.S. Application Data
(63) Continuation of application No. 09/090,757, filed on Jun. 4, 1998.

(51) Int. Cl. A61K 39/395

(52) U.S. Cl. 424/146.1

(57) ABSTRACT

The invention is directed to a method for inhibiting angiogenesis and thus treating inflammatory diseases (e.g., rheumatoid arthritis, diabetic retinopathy, psoriasis, and macular degeneration), inhibiting and preventing tumor growth and preventing metastases in a mammal comprising administering to the mammal an effective amount of a monoclonal antibody which is an antagonist of the integrins, GPIIb/IIIa (α_{IIb}β₃) and αᵥβ₃.

This is a continuation application of U.S. patent application Ser. No. 09/090,757, filed on Jun. 4, 1998.

This invention was made with Government Support under grant #HL19278, awarded by the National Institutes of Health. The government has certain rights in this invention.

### 1. FIELD OF THE INVENTION

The invention is directed to novel methods of use of a monoclonal antibody which inhibits platelet function and also acts as an antagonist of the integrins GPIIb/IIIa(α_{IIb}β₃) and αᵥβ₃. One method of use includes inhibiting angiogenesis and thus angiogenic diseases such as rheumatoid arthritis, psoriasis, diabetic retinopathy, and macular degeneration. Other novel methods of use include preventing and inhibiting tumor growth and metastases.

### 2. BACKGROUND OF THE INVENTION

### 2.1. 7E3 and c7E3 Monoclonal Antibodies

7E3 is a monoclonal antibody that has been well defined in published studies (Coller, 1985, J. Clin. Invest. 76:101-108 and Coller, 1986, J. Cell Biol. 103:451-456). This antibody has been found to inhibit platelet aggregation via its binding to platelet GPIIb/IIIa (α_{IIb}β₃) receptors and prevent thrombus formation. It has also been found that 7E3 binds to αᵥβ₃. receptors (Marcinkiewicz et al., 1996, Biochem. J. 317: 817-825) and prevents the attachment and spreading of endothelial cells on extracellular matrix protein ligands for αᵥβ₃ (Tam et al., 1998, Circulation, in press and Varner et al., 1997, Thromb. Haem. 77(Suppl.) 158).

7E3 is the forerunner of the murine/human chimeric antibody Fab fragment (c7E3 Fab; abciximab; ReoPro™) that is approved for human use in the prevention of ischemic complications of percutaneous coronary interventions. C7E3 Fab has also been found to bind to both GPIIb/IIIa (α_{IIb}β₃) and αᵥβ₃ receptors (Reverter et al., 1996, J. Clin. Invest. 98:863-874 and Nakada et al., 1997, J. Amer. Col. Cardiol. 29:243A).

### 2.2. Angiogenesis

Angiogenesis is the development of new capillaries from preexisting blood vessels (reviewed in Weinstate-Saslow, 1994, The FASEB Journal 8:402-407 and Folkman et al.; 1987, Science 235:442-447). A number of factors are capable of stimulating an angiogenic response. Examples include aFGF, bFGF, TGFα:, TGFβ, EGF, and angiogenin. Some of these factors act directly on endothelial cells, whereas others activate local inflammatory cells to induce angiogenesis. Angiogenesis can have both beneficial and detrimental effects. It can, on the one hand, facilitate wound healing. However, on the other hand it may cause such inflammatory diseases as rheumatoid arthritis, macular degeneration, psoriasis, and diabetic retinopathy.

### 2.3. Tumor Growth and Metastases

The invasive growth and widespread dissemination of human solid tumor cancers remains a problem of great clinical significance. Novel clinical therapies that inhibit tumor proliferation, invasion, and metastasis could improve the outcome of solid tumor cancers.

Tumor-associated angiogenesis is a potential target for such therapy since it has been implicated not only in the growth of tumors but also in their metastasis (Liotta et al., 1991, Cell 64:327; Weinstat-Saslow et al., 1994, FASEB J 8:401; Blood et al., 1990, Biochim. Biophys. Acta 1032:89; Folkman, 1992, Semin. Cancer Biol. 3:65; and Weidner et al., 1991, N. Engl. J. Med. 324:1). Tumor-induced angiogenesis is initiated by growth factors and cytokines that are released from the tumor or from inflammatory cell infiltrates (Brown et al., 1993, Am. J. Path. 143:1255; Brown et al., 1995, Human Path. 26:86; Leek et al., 194, J. Leukocyte Biol. 56:423; Hatva et al., 1995, Am. J. Pathol. 146:368; and Plate et al., 1992, Nature 359:845). Growth factors and cytokines which are expressed by tumor cells stimulate angiogenesis in a number of animal models including the chick chorioallantoic membrane model, the corneal pocket angiogenesis model, and models involving spontaneous and xenotransplanted tumor growth (Brooks et al., 1994, Cell 79:1157; Brooks et al., 1994, Science 264:569; Brooks et al., 1995, J. Clin. Invest. 96: 1815; and Friedlander et al., 1995, Science 27:1500).

Recently, a number of agents have been found to inhibit angiogenesis and tumor growth. Examples include endostatin (O'Reilly et al., 1997, Cell 88:277); angiostatin (O'Reilly et al., 1994, Cell 79:315); the peptide CNGRCVSGCAGRC (SEQ ID NO:1; Arap et al., 1998, Science 279:377); the cyclic peptide RGDfV (SEQ ID NO:2; Friedlander et al., 1995, Science 270:1500); and the monoclonal antibodies LM609 and P1F6 (Friedlander et al., 1995, Science 270:1500). However, the mechanism of action and the source of these angiogenic inhibitors does vary. For example, angiostatin is a 38 kD protein that is obtained from the serum and urine of tumor-bearing mice that inhibits endothelial proliferation; endostatin is a 20 kD protein that is obtained from hemangioendothelioma cells.

The antibody LM609 is an antagonist of the integrin αᵥβ₃ and the antibody P1F6 is an antagonist of the integrin αᵥβ₅. Integrins are a family of heterodimeric proteins that are responsible for such cellular adhesive functions as cell-cell and cell-matrix interactions. It appears that the distribution and functional diversity of integrins is determined by their particular α/β subunit composition.

Other targets for therapy are tumor metastases. For metastases to occur a tumor cell(s) must be able to leave the primary tumor, enter the circulation, invade a second site and proliferate as a secondary colony. The mechanisms of the generation of tumor metastases are unclear. It is thought that the generation of metastases involves a cascade of linked sequential steps involving many host-tumor interactions (reviewed in Liotta et al., 1991, Cell 64:327-336). It has been speculated that platelets may be involved in the formation of metastases. However, mixed results have been obtained with anti-platelet aggregating agents such as aspirin (summarized in Karpatkin et al., 1988, J. Clin. Invest. 81:1012-1019). It has been hypothesized that platelets appear to contribute to metastases by their adhesive interaction with tumor cells via the adhesive proteins fibronectin and von Willebrand factor (Karpatkin et al., 1988, J. Clin. Invest. 81:1012-1019).

As described above, substances known in the art either inhibit angiogenesis and primary tumor growth or prevent metastases. Therefore, it is necessary to administer one course of treatment to inhibit the growth of the primary tumor and another course of treatment to prevent metastases. It would be useful if there was a substance that acts to inhibit angiogenesis and tumor growth as well as prevents metastases. Therefore, it is an object of the invention to formulate a substance that can be used for both purposes.

### 3. SUMMARY OF THE INVENTION

The invention is directed to methods for inhibiting angiogenesis and tumor growth as well as preventing tumor growth or metastases in a mammal in need thereof comprising administering to said mammal a monoclonal antibody or fragment thereof which acts as an antagonist of the integrins GPIIb/IIIa (α_{IIb}β₃) and αᵥβ₃ in an amount effective to inhibit angiogenesis or tumor growth or in an amount effective to prevent tumor growth or metastases in said mammal. The fragment may be an Fab, Fab′ or (Fab′)₂ fragment or derivative thereof (e.g., chimeric, humanized, etc.). The mammal is preferably a primate or dog or cat and most preferably a human patient.

In a specific embodiment, the monoclonal antibody has the identifying characteristics of 7E3 which is produced by the ATCC 8832 hybridoma cell line. Alternatively, the monoclonal antibody may be a chimeric mouse/human monoclonal antibody which comprises a variable or antigen binding region of non-human origin and a constant region of human origin and has the identifying characteristics of the monoclonal antibody, c7E3.

### 4. BRIEF DESCRIPTION OF THE FIGURES

FIG. 1: Human tumor culture in human skin/SCID mouse model. In the human skin/SCID mouse model, six week old SCID mice were engrafted with an 8 mm×13 mm piece of human neonatal foreskin. Four weeks later, the human skin was inoculated with three million human M21L melanoma cells, which were deficient in the expression of the integrin, αᵥβ₃ . After two weeks, mice with palpable tumors were treated either by intravenous or intraperitoneal injections three times per week for three weeks with antibodies or saline. Tumors were excised and analyzed for tumor size and weight.

FIGS. 2A-(1-3): Human tumor angiogenesis in the human skin/SCID mouse model. Tumors were removed from human skin grafts bearing tumors, embedded in OCT and snap frozen. Five micron cryostat sections of tumors were fixed in acetone and stained by indirect immunofluorescence with anti-human CD31 (PECAM FIG. 2A-2), a marker of endothelial cells (red) or with anti-von Willebrand factor (FIG. 2A-1), a marker of human and mouse blood vessels (green). Colocalization of von Willebrand factor and human CD31 can be observed by merging the two images (yellow: FIG. 2A-3). Blood vessels which are positive for both markers are of human origin (arrows).

FIGS. 3A-B: 7E3 inhibits human tumor growth in human skin/SCID mouse model. Six week old SCID mice were engrafted with an 8 mm×13 mm piece of human neonatal foreskin. Four weeks later, the human skin was inoculated with three million human M21L melanoma cells. After two weeks, mice with palpable tumors were treated by intravenous or intraperitoneal injections three times per week for three weeks with 300 μg doses of 7E3, AP3 (an isotype matched control antibody) or saline. Tumors were excised and analyzed for tumor size (FIG. 3A) and weight (FIG. 3B).

FIGS. 4A-B: 7E3 inhibits human tumor growth in human skin/SCID mouse model as well as LM609. Six-week old SCID mice were engrafted with an 8 mm×13 mm piece of human neonatal foreskin. Four weeks later, the human skin was inoculated with three million human M21L melanoma cells. After two weeks, mice with palpable tumors were treated either by intravenous or intraperitoneal injections three times per week for three weeks with 300 mg doses of 7E3, LM609 (an anti-αᵥβ₃ specific monoclonal antibody), an isotype-matched control anti-b₃ antibody or saline. Tumors were excised and analyzed for tumor volume (FIG. 4A) and weight (FIG. 4B).

FIG. 5: Both 7E3 and LM609 significantly inhibit tumor growth. A comparison of tumor volumes and weights for three separate experiments are shown. Using the exact Wilcox rank-sum test, stratifying on experimental groups, both the LM609 and 7E3 treated groups had smaller tumor volumes (p=0.003 and p<0.0001, respectively) and tumor weights (p=0.02 and p=0.0007, respectively) than control treated animals.

FIGS. 6(A-1-3), (B1-3), (C): 7E3 and LM609 inhibit human angiogenesis. Tumors treated with saline, 7E3 or LM609 were removed from the human skin grafts, embedded in OCT and snap frozen. Five micron cryostat sections were fixed in acetone and stained by indirect immunofluorescence with anti-human CD31 (A-1=saline treated: A-2=LM609 treated: and A-3=7E3 treated) or with anti-murine CD31 (B-1=saline treated: B-2=LM609 treated: and B-3=7E3 treated). Tissue sections were photographed at a magnification of 200 × (C). Blood vessels staining positive for human CD31 were quantified on 15 randomly selected microscopic fields at 200×.

### 5. DETAILED DESCRIPTION OF THE INVENTION

### 5.1. Preparation of Monoclonal Antibodies

The monoclonal antibody used in the method of the present invention may be prepared using procedures known in the art.

In a specific embodiment, the monoclonal antibody has the identifying characteristics of 7E3.The procedure described in U.S. Pat. No. 5,440,020 may be used to obtain said monoclonal antibody. Specifically, human blood platelets are injected into mice. The mouse spleen is removed and fused with mouse myeloma cells by a modification of the technique of Levy et al., 1984, Curr. Top. Microbiol. Immunol. 81: 164. The fused cells are incubated and then further incubated in HAT medium. The cells are then diluted out and screened in a screening assay for antifibrinogen receptor activity (Coller et al., 1983, J. Clin. Invest. 72:3235-338). There is selected a clone which reacts with normal human blood platelets and with dog blood platelets, fails to react with thrombasthenia platelets or human platelets whose GPIIb/IIIa complex was dissociated with EDTA, reacts slowly with unactivated human platelets and more rapidly with ADP activated human platelets and blocks the interaction of fibrinogen with platelets induced by ADP (Coller et al., 1983, J. Clin. Invest. 72:325-338). Additionally, the antibody acts as an antagonist to the integrin αᵥβ₃ by inhibiting the binding of extracellular matrix ligands to integrin αβ₃ and preventing the αᵥβ₃-dependent attachment of cells to extracellular matrix protein ligands. The binding of the antibody to αᵥβ₃ receptors may be detected, for example, by platelet adhesion assays as described in Coller et al., 1991, Blood 77:75-83. The antibody is subsequently isolated from the supernatant in wells or flasks.

Alternatively, the monoclonal antibody may be obtained from a hybridoma having the identifying characteristics of the ATCC HB 8832 hybridoma cell line. The hybridomas are injected intraperitoneally into Pristane pretreated BALB/c mice and the antibodies are isolated from the ascitic fluid. The antibody is purified by precipitation with 50% saturated ammonium sulfate, resuspended in between 5 and 10% of the original volume in sodium phosphate buffer and dialyzed against the same buffer. Chromatography on protein A-SEPHAROSE CL-4B equilibrated with phosphate buffer is carried out, elution is with phosphate buffer followed by decreasing pH 0.1M citrate buffers. 7E3 antibody is eluted after the pH decreased to about 6.0. Protein elution is monitored by ultraviolet spectroscopy at 280 nm. Purified antibody is then dialyzed overnight at reduced temperatures, suitably between about 0° C. and 10° C., preferably at 4° C. against a slightly acidic saline buffer of pH 3.5-6.5, suitably about pH 4.0.

Antibody fragments may be obtained according to methods known in the art. Such fragments include but are not limited to: the F(ab′)₂ fragment which can be generated by treating the antibody molecule with pepsin; the Fab′ fragments which can be generated by reducing the disulfide bridges of the F(ab′)₂ fragment; and the 2Fab or Fab fragments which can be generated by treating the antibody molecule with papain and a reducing agent to reduce some of the disulfide bridges.

In another specific embodiment, the monoclonal antibody may have the identifying characteristics of the chimeric human/mouse antibody c7E3 which is also an antagonist of αᵥβ₃ and (GPIIb/IIIa)α_{IIb}β₃. The heavy chain comprises an antigen-binding region drived from the heavy chain of a non-Human antibody specific for platelets (e.g., specific for the GPIIb/IIIa receptor) linked to a human heavy chain constant region. The light chain comprises an antigen binding region derived from the light chain of the non-human antibody linked to a human light chain constant region.

This antibody may be obtained using procedures known in the art (see, for example, PCT appln no. PCT/US94/12779). Specifically, the chimeric antibodies are produced by preparing for each of the light and heavy chain components a fused gene comprising a first DNA segment that encodes at least the functional portion of the platelet-specific variable region of nonhuman origin linked to a second DNA segment encoding at least a part of a human constant region. Each fused gene is assembled in or inserted into an expression vector. Host cells are then transfected with the expression vector. The transfected host cells are cultured under conditions which promote expression of the DNA sequences encoding the chimeric antibody and the expressed antibody is recovered.

In a preferred embodiment, an Fab fragment is used. Said fragment may be purchased from Centocor and goes by the tradename ReoPro™ or generated using procedures described supra.

### 5.2. Compositions and Their Uses

The monoclonal antibody described supra can be used to inhibit angiogenesis and thus treat inflammatory diseases which may include but are not limited to rheumatoid arthritis, diabetic retinopathy, psoriasis, and macular degeneration. Additionally, said monoclonal antibody can be used to inhibit and prevent tumor growth, and prevent metastases. The individual to be treated may be any mammal and is preferably a primate, a dog or cat and most preferably a human patient. The amount of monoclonal antibody administered will vary according to the purpose it is being used for and the method of administration. Administration may also be oral or by local injection into a tumor or tissue. In a preferred embodiment, the monoclonal antibody is administered intravenously. Generally, the dosage range is 0.25 mg/kg as a bolus dose. This may be followed by a continuous infusion of 0.125 mg/kg/min for about 3 weeks.

Alternatively, DNA encoding preferably a fragment of said monoclonal antibody may be isolated from hybridoma cells and administered to a mammal. The DNA may be administered in naked form or inserted into a recombinant vector, e.g., vaccinia virus.

The monoclonal antibody used in the method of the present invention may be formulated by any of the established methods of formulating pharmaceutical compositions, e.g. as described in Remington's Pharmaceutical Sciences, 1985. For ease of administration, the monoclonal antibody will typically be combined with a pharmaceutically acceptable carrier. Such carriers include water, physiological saline, or oils. Except insofar as any conventional medium is incompatible with the active ingredient and its intended use, its use in any compositions is contemplated.

### 6. EXAMPLE

### 6.1. Materials and Methods

### 6.1.1. Antibodies and Reagents

Murine 7E3, was produced at Centocor, Malvern, Pa. LM609 was the kind gifts of Dr. David Cheresh. AP3 was a gift from Peter Newman, Blood Research Institute, Milwaukee, Wis. Anti-human CD31 was purchased from Chemicon International (Temecula, Calif.). Anti-murine CD31 was purchased from Pharmingen (San Diego, Calif.). Anti-von Willebrand factor was purchased from Biogenex (San Ramon, Calif.). Cross-absorbed secondary antibodies were purchased from Chemicon International or from Biosource International (Camarillo, Calif.). Six-week old CB17 female SCID mice were purchased from Charles River. Fresh human neonatal foreskins were obtained from the Cooperative Human Tissue Network of the National Institutes of Health and were stored in RPMI-1640 media supplemented with 2% fetal bovine serum and 1% gentamicin. Human M21L melanoma cells (Fielding-Habermann et al., 1992, J. Biol. Chem. 267:5070-507) were a gift from Dr. David Cheresh. OCT embedding medium was obtained from Baxter (McGraw Park, Ill.). Fluoromount embedding medium was purchased from Southern Biotechnology Associates (Birmingham, Ala.).

### 6.1.2. Cell Culture

M21L human melanoma cells which were deficient in αᵥβ₃ were routinely cultured in RPMI medium (Irvine Scientific, Irvine, Calif.) supplemented with 10% fetal bovine serum, glutamine, and 1% gentamicin. To prepare cells for inoculation into the transplanted human skin on SCID mice, tumor cells were removed from culture dishes by brief treatment with EDTA, washed once briefly in complete medium and then thoroughly in saline. Tumor cells were resuspended and fifty microliters containing three million cells were injected intradermally in the center of the human skin.

### 6.1.3. SCID Mouse Model of Human Tumor Growth

In this model, tumors were primarily supplied by human blood vessels derived from the human skin, but large tumors eventually recruit some murine blood vessels. This model has the further advantage of essentially excluding the possibility that 7E3 may be working through its antiplatelet effects since 7E3 does not react with murine platelets.

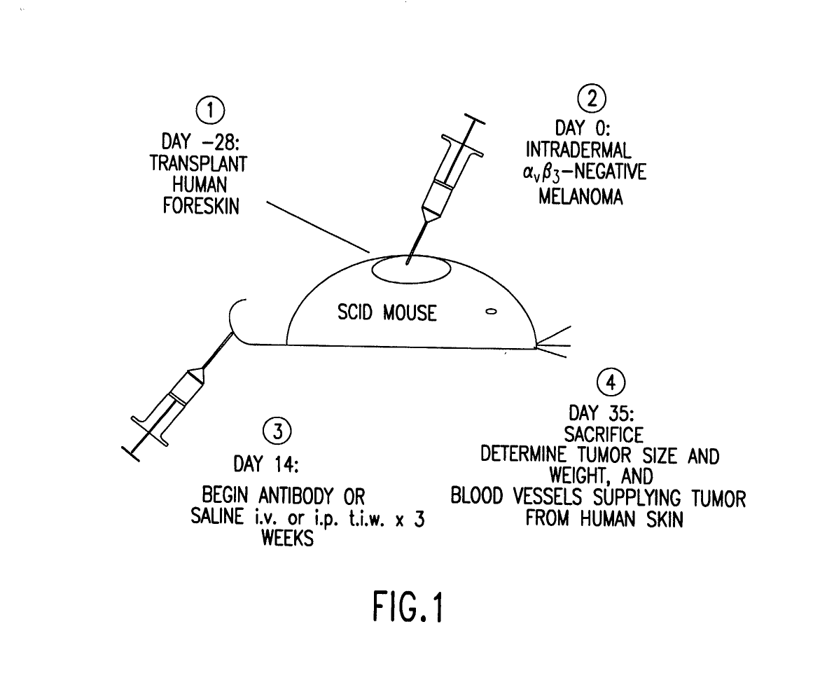

Engraftment of SCID mice with human skins was performed as described (Brooks et al., 1995, J. Clin. Invest. 96: 1815). The general procedure used is shown in FIG. 1. Six-week old SCID mice were engrafted with an 8 mm×13 mm piece of human neonatal foreskin. Four weeks later, the human skin was inoculated with three million human M21L melanoma cells. After two weeks, mice with palpable tumors were treated either by intravenous or intraperitoneal injections three times per week for three weeks with 50 μl of saline or 300 μg doses of 7E3, LM609 (an anti-αᵥβ₃ murine monoclonal antibody) or an isotype-matched control murine monoclonal antibody. At the end of the three week treatment period, tumors were excised and analyzed for tumor size using calipers and for wet weight. Tumor volumes were calculated using the formula (w²×1)/2 (w is width and 1 is length).

### 6.1.4. Immunohistochemistry and Quantification of Blood Vessels

Tumors were removed from human skin grafts bearing M21L tumors, embedded in OCT and snap frozen. Five micron cryostat sections of tumors were fixed for one minute in acetone and stained by indirect immunofluorescence by incubating sections in 2% bovine serum albumin in saline for two hours followed by incubation of sections in 5 μg/ml of murine anti-human CD31 (PECAM), rat anti-murine CD31,or rabbit anti-von Willebrand factor. Sections were washed five times with saline and incubated for 1 hour in goat anti-rabbit-IgG-FITC or goat anti-mouse-IgG-rhodamine. Sections were washed five times with saline and mounted with Fluoromount. Two color staining for colocalization of von Willebrand factor and human CD31 was performed essentially as described (Brooks et al., 1995, J. Clin. Invest. 96: 1815). Blood vessels staining positive for human CD31 were quantified on 15 randomly selected microscopic fields at 200×.

### 6.1.5 Statistical Analysis

Statistical analyses were performed using StatXact 3 for Windows, CYTEL software Corporation, Cambridge, MA.

### 6.2. Results

### 6.2.1. Growth of Human Melanoma in the SCID Mouse/Human Skin Model of Human Angiogenesis

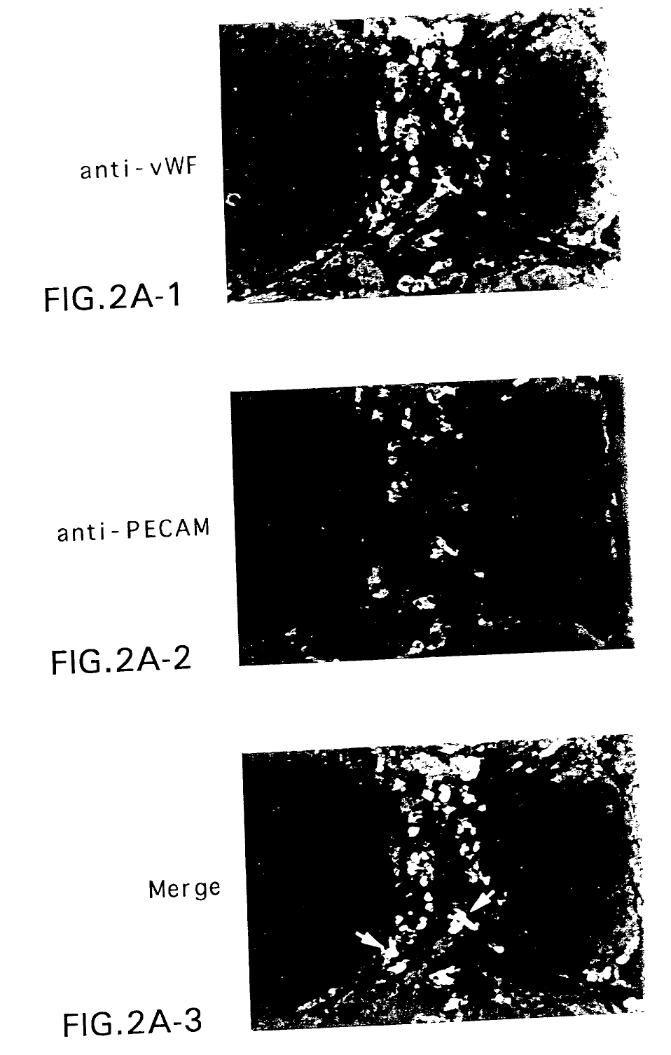

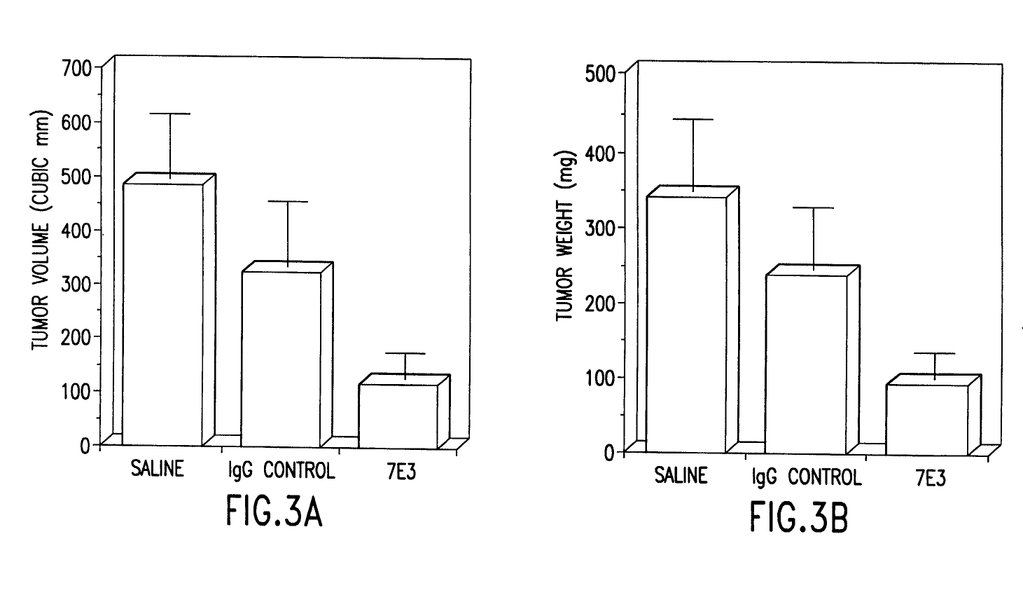

To assess whether M21L tumors were supplied by human blood vessels derived from the human skin or mouse blood vessels infiltrating the human skin graft, control tumors were excised four weeks after inoculation of tumor cells (see FIG. 1) and frozen sections from these tumors were stained with an antibody specific for human CD31 (PECAM), a constitutive marker of adult endothelial cells (DeLisser et al, 1994, Immunol. Today 15:490-495) and with an antibody directed against both mouse and human von Willebrand factor, an extracellular matrix protein marker of mature blood vessels. The majority of the blood vessels identified by either hematoxylin and eosin staining or by immunofluorescence analysis were associated with the edges of nests of M21L tumor cells (FIGS. 2A-1-3). Blood vessels with both large and small lumens stained positively for von Willebrand factor (FIG. 2A-1); vessels with small lumens were positive for human CD31 (FIG. 2A-2). When the anti-CD31 and anti-von Willebrand factor images were merged to identify the overlap in expression of these two markers (seen as yellow; FIG. 2A-3), the majority of the vessels were shown to be positive for both human CD31 and von Willebrand factor expression (arrows). Approximately 10% of the vessels were positive only for von Willebrand factor. Since the antibody against von Willebrand factor reacts with both mouse and human blood vessels, these results indicate that the majority of the blood vessels in the tumor grown in the human skin graft are of human origin, but˜10% are of murine origin.

### 6.2.2. 7E3 Inhibits Tumor Growth

To evaluate the effect of 7E3 on human angiogenesis and tumor growth, tumor-bearing mice were injected either intravenously or intraperitoneally three times a week for three weeks with 300 μg of 7E3, a control isotype-matched anti-integrin β3 antibody, AP3 or with saline (FIGS. 3A-B). While both 7E3 and AP3 react with integrins αᵥβ₃ and α_{IIb}β₃, only 7E3 inhibits the function of these integrins. Mice were treated three times a week for three weeks with 300 μg doses of each antibody. Tumor dimensions were measured with calipers at the end of the three weeks of treatment. Tumors were then resected and wet weights determined. Systemic administration of 7E3 resulted in a significant reduction in both tumor weight and tumor volume when compared to control treatments (FIGS. 3A-B). Tumors from 7E3 treated mice were 60% smaller on the basis of tumor weight (FIG. 3B) and 64% smaller on the basis of tumor volume (FIG. 3A) when compared to tumors treated with the control antibody AP3. 7E3 treatment resulted in a 72% reduction in tumor weight (FIG. 3B) and a 75% reduction in tumor volume (FIG. 3A) when compared to tumors from saline treated mice.

### 6.2.3. 7E3 Prevents Tumor Growth as well as L609

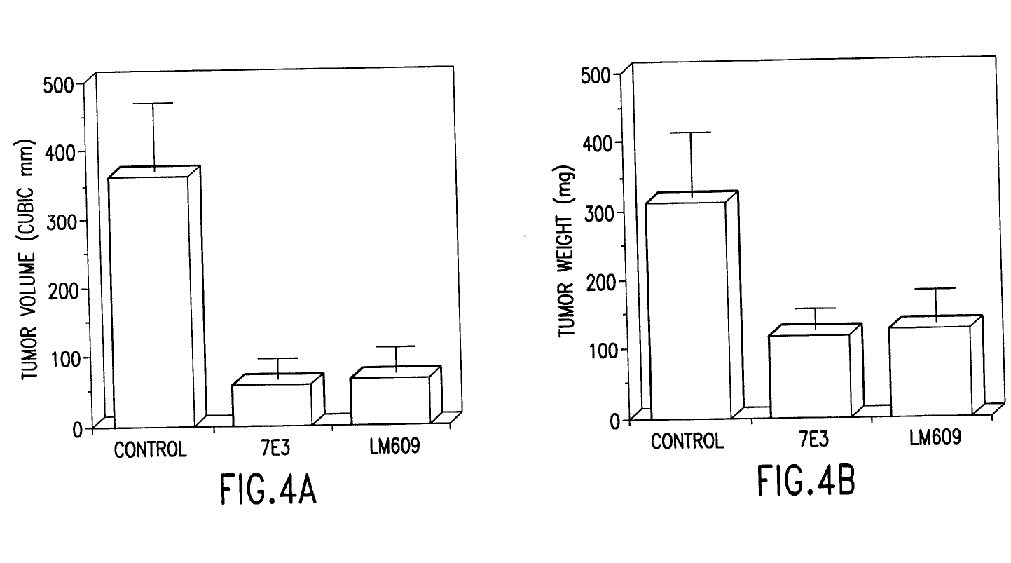

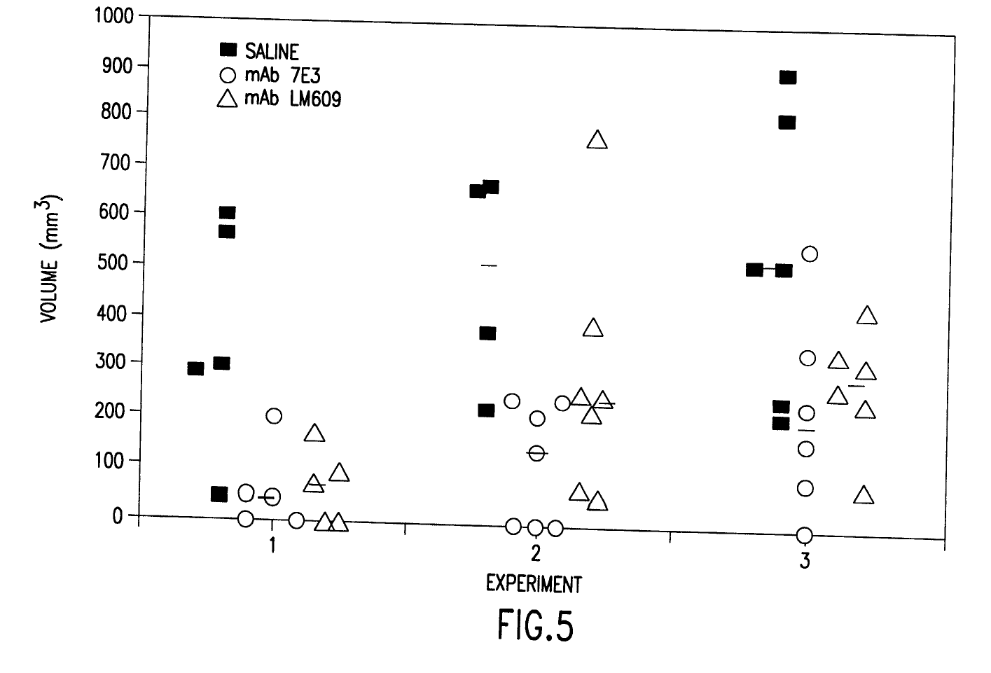

To further evaluate the efficacy of 7E3 to inhibit tumor growth, the potency of 7E3 was compared to that of LM609, a specific monoclonal antibody antagonist of integrin αᵥβ₃ with demonstrated anti-angiogenic properties (Brooks et al., 1994, Cell 79:1157; Brooks et al., 1994, Science 264:569; Brooks et al., 1995, J. Clin. Invest. 96: 1815; and Friedlander et al., 1995, Science 27:1500). Thrice weekly intravenous or intraperitoneal injections of 300 μg of 7E3, 300 mg of LM609 or of an equivalent volume of saline were administered to groups of mice bearing palpable tumors normalized for size. As seen in FIGS. 4A-B, 7E3 was as effective as LM609 in inhibiting tumor growth in the SCID mouse/human skin model. In this experiment, 7E3 and LM609 inhibited tumor volumes by 85% and 82% respectively (FIG. 4A), and tumor weights by 64% and 60% respectively (FIG. 4B). Studies comparing 7E3 to LM609 were performed three times with six to eight mice per treatment group. In each experiment, treatment of mice bearing tumors with either 7E3 or LM609 resulted in statistically significant suppression of tumor growth as evaluated either by tumor volume or tumor weight (FIG. 5). Using the exact Wilcox on rank-sum test, stratifying on experimental groups, both the LM609-treated and 7E3-treated animals had significantly smaller tumor volumes than did the saline control group (p=0.003 and p<0.0001). Similar results were obtained using tumor weights instead of tumor volumes. Both the LM609-treated and 7E3 treated animals had significantly lighter weight tumors than the saline-treated animals (p=0.02 and p=0.0007).

### 6.2.4. 7E3 Inhibits Tumor-Induced Angiogenesis

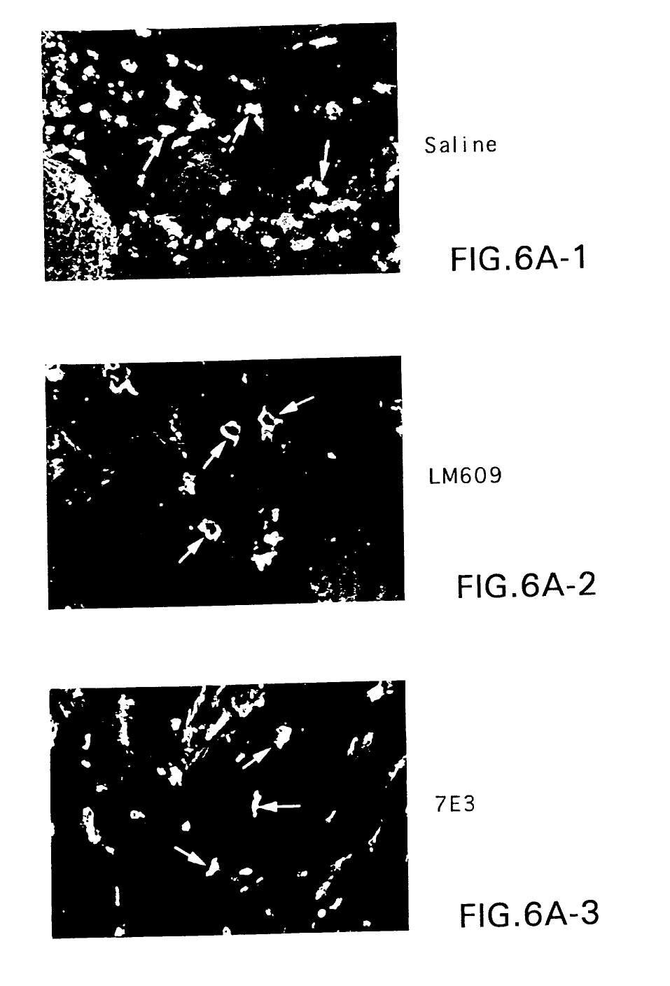

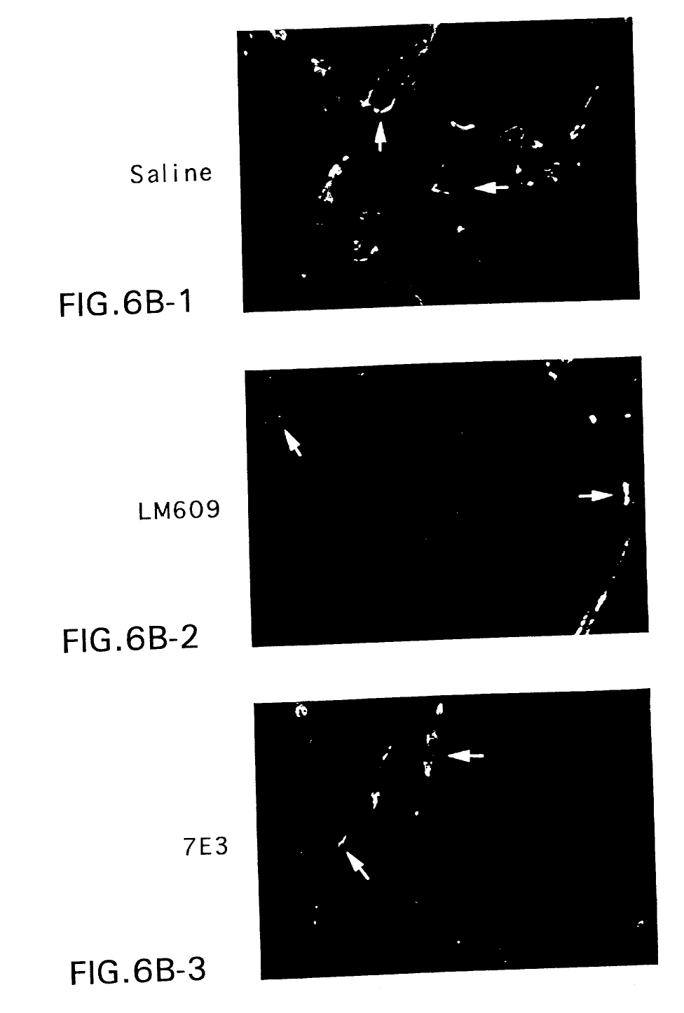

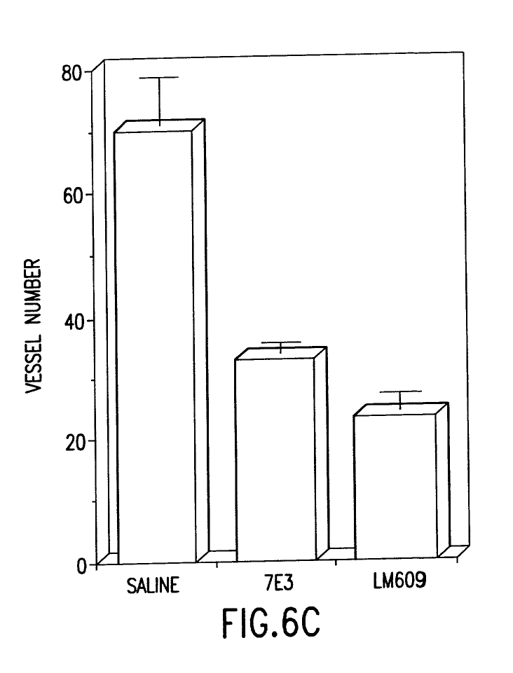

Histological analysis of 7E3 treated tumors demonstrated that 7E3 significantly inhibits human angiogenesis. As shown in FIGS. 6A(1-3), tumors from mice treated with either saline or control IgG have numerous human CD31 positive blood vessels. Tumors from mice treated with 7E3 or LM609 exhibit a significant reduction in human CD31 positive blood vessels. When human CD31 positive vessels were quantified by counting the number of vessels per high power (200×) microscopic field in fifteen representative microscopic fields, an average of 70 vessels per field were observed in saline treated tumors. In contrast, treatment with 7E3 resulted in an average of 33 vessels per section and LM609 resulted in an average of 23.5 vessels per microscopic field (FIGS. 6[B]C).

When tumor sections were stained for the presence of murine blood vessels by staining frozen sections with an antibody that is specific for murine CD31 (FIGS. 6B(1-3)), significantly fewer murine vessels were present in 7E3and in LM609- treated tumors than in control-treated tumors (FIG. 6C). Since 7E3 and LM609 do not react with murine integrins, these results suggest that in this model, angiogenesis proceeds in two phases, the first in which human blood vessels supply the tumor, and the second, in which the large tumor can recruit blood vessels from the surrounding murine tissues. By inhibiting the first phase of angiogenesis dependent on the human blood vessels, 7E3 and LM609 prevent the tumor from growing to a size capable of recruiting murine blood vessels.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Various references are cited herein, the disclosures of which are incorporated by reference in their entireties.

What is claimed is:

1. A method for inhibiting angiogenesis in a mammal in need thereof comprising administering to the mammal a monoclonal antibody or fragment thereof which acts as an antagonist of the integrins GPIIb/IIIa (α_{IIb}β₃) and αᵥβ₃ in an amount effective to inhibit angiogenesis in said mammal.

2. The method according to claim 1, in which the antibody fragment is an Fab, Fab′, or F(ab′)2 fragment or derivative thereof.

3. The method according to claim 1, in which the monoclonal antibody has the identifying characteristics of monoclonal antibody 7E3, produced by the ATCC 8832 hybridoma cell line.

4. The method according to claim 1, in which the monoclonal antibody has the identifying characteristics of monoclonal antibody c7E3.

5. The method according to claim 1, in which the monoclonal antibody is administered intravenously.

6. The method according to claim 1, in which the monoclonal antibody is administered in the amount of about 0.25 mg/kg body weight.

7. The method according to claim 1, in which the monoclonal antibody is administered in the amount of about 0.25 mg/kg body weight followed by an infusion of 0.125 mg/kg/min of said antibody.

8. The method according to claim 1, in which the mammal is selected from the group consisting of a primate, dog, cat, and human.

9. The method according to claim 1, in which the mammal is a human patient.

10. The method according to claim 1, in which said monoclonal antibody treats an inflammatory disease.

11. The method according to claim 1, in which said monoclonal antibody treats an inflammatory disease selected from the group consisting of rheumatoid arthritis, macular degeneration, psoriasis, diabetic retinopathy.

12. A method for inhibiting tumor growth in a mammal in need thereof comprising administering to the mammal a monoclonal antibody or fragment thereof which acts an antagonist of the integrins, GPIIb/IIIa (α_{IIb}β₃) and αᵥβ₃ in an amount effective to inhibit the growth of said tumor.

13. A method for preventing tumor growth in a mammal in need thereof comprising administering to the mammal a monoclonal antibody or fragment thereof which acts as an antagonist of the integrins GPIIb/IIIa (α_{IIb}β₃) and αᵥβ₃ in an amount effective to prevent the growth of said tumor in said mammal.

14. A method for preventing metastases in a mammal in need thereof comprising administering to the mammal a monoclonal antibody or fragment thereof which acts as an antagonist of the integrins GPIIb/IIIa (α_{IIb}β₃) and αᵥβ₃ in an amount effective to prevent metastases in said mammal.
